(19) United States
(12) Patent Application Publication
(10) Pub. No.: US 2002/0058990 A1
(43) Pub. Date: May 16, 2002

(54) Intravascular stent consists of stairstep expansion strut pairs and double stairstep diagonal connecting struts contralaterally extended from expansion struts

(76) Inventor: G. David Jang, Redlands, CA (US)

Correspondence Address: WILSON SONSINI GOODRICH & ROSATI, 650 PAGE MILL ROAD, PALO ALTO, CA, 943041050

(21) Appl. No.: 09/942,077

(22) Filed: Aug. 28, 2001

Related U.S. Application Data
(60) Provisional application No. 60/235,164, filed on Sep. 23, 2000.

(51) Int. Cl. A61F 2/06

(52) U.S. Cl. 623/1.15

(57) ABSTRACT

An intra-coronary stent provides intraluminal-scaffolding support of a vascular wall after percutaneous angioplasty in which a balloon catheter is used to expand a stenotic vascular lesion. The flexible stent substantially fully covers a vessel surface inside a vascular lumen. The improved stent easily delivers a delivery balloon to a selected vascular lumen site. The flexible stent has a smooth surface and sufficient radiopacity in the delivery and post delivery phase.

### CROSS-REFERENCE TO RELATED APPLICATIONS

This application claims the benefit of Provisional Patent Application No. 60/235,164 filed Sep. 23, 2000, the disclosure of which is incorporated by reference.

### FIELD OF INVENTION

This invention relates generally to intravascular stents, and more particularly to intra-coronary stents that provide intraluminal-scaffolding support of a vascular wall after percutaneous angioplasty in which a balloon catheter is used to expand a stenotic vascular lesion.

### DESCRIPTION OF THE RELATED ART

By the year 2000, the percutaneous balloon angioplasty and stent implant procedures have become the dominant non-surgical revascularization method of the atherosclerotic stenosis, or obstruction, of the vascular lumen, and particularly in the coronary vascular system of the heart. With balloon angioplasty alone, without use of stent, the restenosis rate after angioplasty has been as high as 25-45% in the first time coronary cases. With use of stents after balloon angioplasty, the restenosis has been reduced significantly. Even so, the restenosis rate after stent implant is reported to be 15-25% range in coronary arteries, depending on the condition of the vessel stented or what specific stent was used. An ideal coronary stent is still elusive in the current state of the art commercial products.

Some of the best selling current, second generation, stents can be divided into two categories. One category is a stent with flexibility and the other with good vessel coverage. The flexible current stents generally have poor vessel coverage, tissue prolapse, rough surface modulation and increased restenosis rate. On the other hand, a good vessel coverage stent in the current state of art has better vessel coverage but not flexible enough for easy delivery and efficient procedure. This means that an ideal stent that has good flexibility and good vessel coverage remains as the gold standard that has not yet been reached.

To further reduce the restenosis rate after stent implant, numerous means has been tried, including laser, atherectomy, high frequency ultrasound, radiation device, local drug delivery, etc. Although the brachytherapy (radiation treatment) has proved to be reasonably effective in further reducing restenosis after stent implant, using brachytherpy is very cumbersome, inconvenient and costly. Mainly because it is radioactive device and radiation therapy specialist from another department has to be involved with the interventional cardiologist in the cardiac catheterization laboratory. The laser and atherectomy devices proved to be marginally useful in this purpose with added costs. Even if these measures would reduce the restenosis rate in theory or in real terms, an ideal stent that has good vessel coverage and flexibility would produce even better outcomes.

The local drug therapy appears to be a very promising method for the future, as better pharmaceutical, chemical or biogenetic agents are developed and became available. Some research data, both from animal tests and human clinical studies, indicate that there are evidences of suppressing restenosis after stent implant when certain growth blocking pharmaceutical agents available today are used to coat the stent. In another instances, it has been speculated that certain surface modifying materials coated on the surface of the stent may be beneficial by it alone or in combination with growth suppressing agent, in reducing restenosis rate. In either instance, the drug or substance should be locally attached or coated on the stent and in sufficient amounts. However, attaching or coating a sufficient amount of a substance or drug on the coronary stent is not so easy a proposition, because coating enough volume of the drug on the small surface area of the stent is an elusive dream. If and when stent coating becomes practical, an ideally designed stent would still have better outcomes than a poorly designed stent, when used with substance coating.

There is a need for an improved stent that is very flexible and substantially fully covers a vessel surface inside a vascular lumen.

### SUMMARY OF THE INVENTION

Accordingly, an object of the present invention is to provide a stent that is very flexible and substantially fully covers vessel surfaces inside a vascular lumen.

Another object of the present invention is to provide an improved stent that is easily delivered with a delivery balloon to a selected vascular lumen site.

Still another object of the present invention is to provide a stent that provides good flexibility, a smooth surface modulation without tulips and sufficient radiopacity during the delivery phase of the stent.

A further object of the present invention is to provide a stent with good flexibility, a smooth surface modulation without tulips and sufficient radiopacity in the delivery and post delivery phase to the selected vascular site.

These and other objects of the present invention are achieved in a stent, in a non-expanded state, that includes a first expansion column with individual expansion struts which form a plurality of expansion strut pairs. Each two adjacent expansion strut pairs in the first expansion column share a common strut. A second expansion column has individual expansion struts that form a plurality of expansion strut pairs. Each two adjacent expansion strut pairs in the second expansion column share a common strut. A first connecting strut column includes a plurality of individual connecting struts. Each individual connecting strut is an extension arm of an individual expansion strut from the first expansion column, and an extension arm of an individual expansion strut of the second expansion column.

In another embodiment of the present invention, a stent, in a non-expanded state, has a first expansion column with individual expansion struts that form a plurality of expansion strut pair loops. The expansion strut pair loops couple adjacent individual expansion struts. Each two adjacent expansion strut pairs in the first expansion column share a common strut. A second expansion column includes individual expansion struts that form a plurality of expansion strut pair loops. The expansion strut pair loops couple adjacent individual expansion struts. Each two adjacent expansion strut pairs of the second expansion column share a common strut. A first connecting strut column has a plurality of individual connecting struts. Each end of an individual connecting strut extends from ipsi-lateral sides of expansion strut pair loops of the first and second expansion columns.

In another embodiment of the present invention, a stent, in a non-expanded state, has a first expansion column with individual expansion struts that form a plurality of expansion strut pair loops. The expansion strut pair loops couple adjacent individual expansion struts. Each two adjacent expansion strut pairs in the first expansion column share a common strut. A second expansion column includes individual expansion struts that form a plurality of expansion strut pair loops. The expansion strut pair loops couple adjacent individual expansion struts. Each two adjacent expansion strut pairs of the second expansion column share a common strut. A first connecting strut column has a plurality of individual connecting struts. Each end of an individual connecting strut extends from contra-lateral sides of expansion strut pair loops of the first and second expansion columns.

In another embodiment of the present invention, a stent, in a non-expanded state, a first expansion column includes individual expansion struts that form a plurality of expansion strut pair loops. The expansion strut pair loops form first and second bifurcated expansion strut arms at expansion strut bifurcation points. Each two adjacent expansion strut pairs in the first expansion column share a common strut. A second expansion column includes individual expansion struts that form a plurality of expansion strut pair loops. The expansion strut pair loops form first and second bifurcated expansion strut arms at expansion strut bifurcation points. Each two adjacent expansion strut pairs share a common strut. A first connecting strut column has a plurality of individual connecting struts. One of the expansion strut arms forms a portion of a connecting strut.

### BRIEF DESCRIPTION OF DRAWINGS

FIG. 1 is a side-elevation view of one embodiment of an unexpanded stent of the present invention.

FIG. 2 is an isometric view of the FIG. 1 stent drawn in scale for a 15 mm length.

FIG. 3 is a close up view of the first and second expansion columns.

FIG. 4 is a close up view of the FIG. 1 stent.

FIG. 5 illustrates one embodiment of the connecting struts of the first connecting strut column.

FIG. 6 illustrates one embodiment of the connecting struts that form the second connecting strut column.connector structs joining the expansion struts of FIG. 4.

FIG. 7 is a close up view of closed cells created by adjacent expansion columns and their associated connecting strut colums.

FIG. 8 is illustrates the alignment of two adjacent expansion columns.

### DETAILED DESCRIPTION OF THE PREFERRED EMBODIMENTS

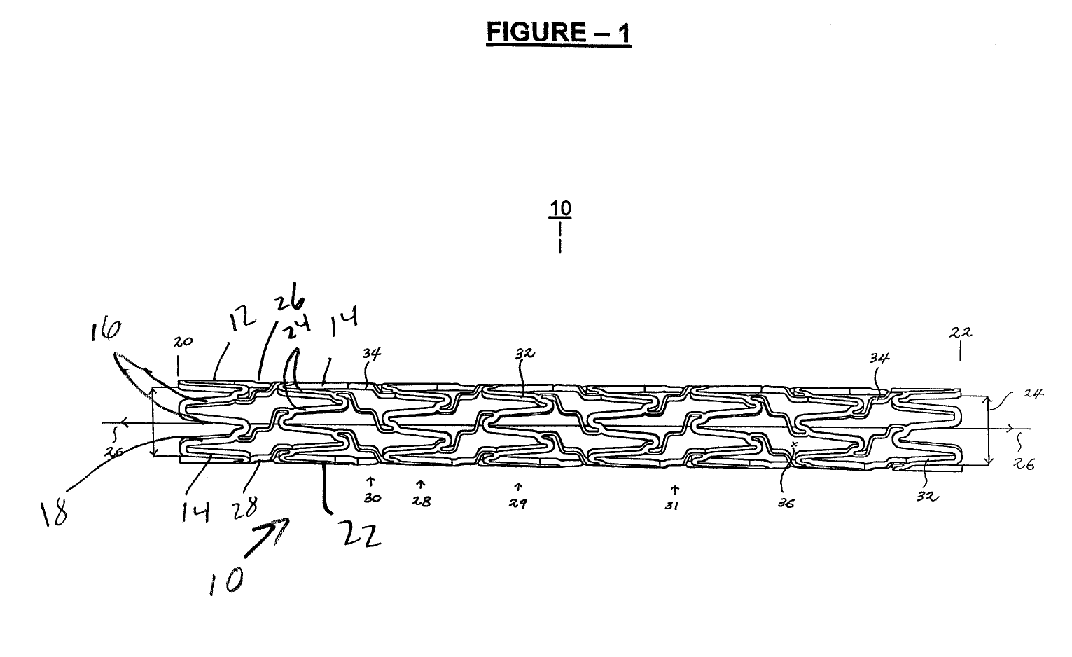

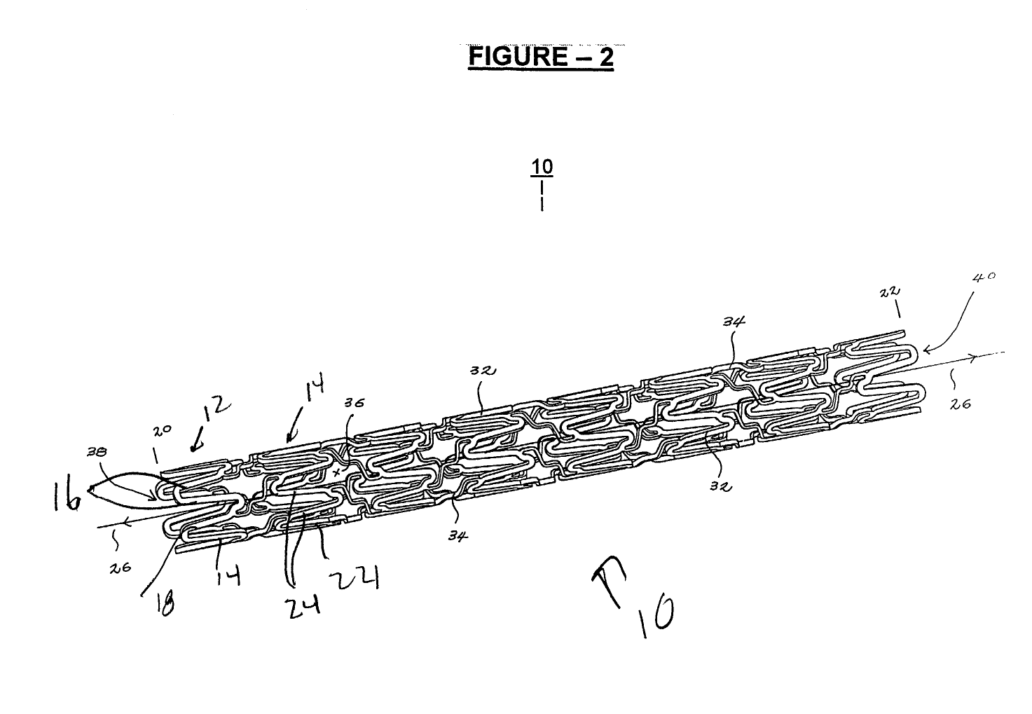

Referring now to FIGS. 1 and 2, one embodiment of a stent 10 of the present invention is illustrated in a non-expanded state. Stent 10 includes a first expansion column 12 with individual expansion struts 14 that form a plurality of expansion strut pairs 16. Adjacent expansion strut pairs 16 in first expansion column 12 share a common strut, indicated as 18. A second expansion column 20 also has individual expansion struts 22 that form a plurality of expansion strut pairs 22. Adjacent expansion strut pairs 22 in second expansion column 20 share a common strut, indicated as 24. Stent 10 is configured to provide easy delivery that is achieved with a sufficient level of flexibility in combination with a delivery balloon, a smooth surface modulation without tulips and a reasonable radiopacity during the delivery phase of stent 10.

Stent 10 provides enhanced flexibility and conformability while maintaining a full vessel coverage with optimal metal fraction. Additionally, stent 10 has, (i) substantially evenly expanding stent struts, (ii) excellent radial strength and radiopacity and (iii) smooth surface modulations in both the delivery and deployed phases of the stent life cycle. Stent 10 has a continuous, unbroken cylindrical form without any break or de-linking around the circumference and along its length.

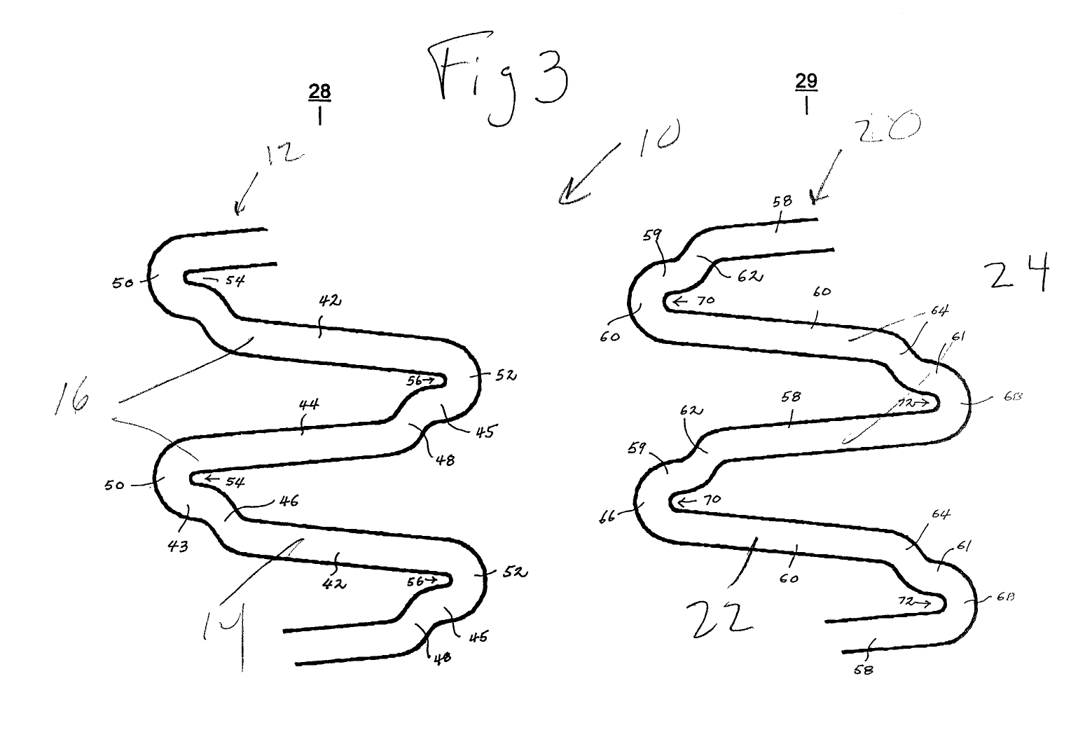

As illustrated in FIG. 3, expansion strut pairs 16 and 24 form loops that couple adjacent individual expansion struts 14 and 22. In one embodiment, the loops of expansion strut pairs 16 and 24 are aligned in a peak to valley geometry. In another embodiment, the loops of expansion strut pairs 16 and 24 are aligned in a peak to peak geometry.

Expansion strut pairs 16 and 24 are jointed by a joining strut segment on distal and proximal ends and form the loops to form a “zigzig” pattern that continues for a selected number of cycles without a break around a circumference of stent 10. The number of cycles can be any number but in one embodiment the number is six or less.

Expansion struts 14 and 22 have first and second segments. At least a portion of the first segment of expansion struts 14 is positioned in close proximity in front of the loop of an expansion strut pair 16. At least a portion of the second section of expansion struts 22 is positioned in close proximity in front of the loop of an expansion strut pair 24. In one embodiment, close proximity is a distance of at least 0.001 inch. In another embodiment, close proximity is a distance less than 0.04 inch. At least one expansion strut 14 and 22 of expansion strut pairs 16 and 24 can have a stair-step segment at its proximal end and, the other expansion strut of the expansion strut pairs 16 and 24 has a stair-step segment at its distal end. In various embodiments, expansion struts 14 and 22 can have a, (i) short stepped-down segment at the proximal end, (ii) short stepped-down segment at the distal end, (iii) short stepped-up segment at the proximal end and short stepped-up segment at a the distal end. In all of these embodiments, expansion struts 14 and 22 have a short sloped transitional segment of that can have the same length between the long and short parts in expansion struts 14 and 22.

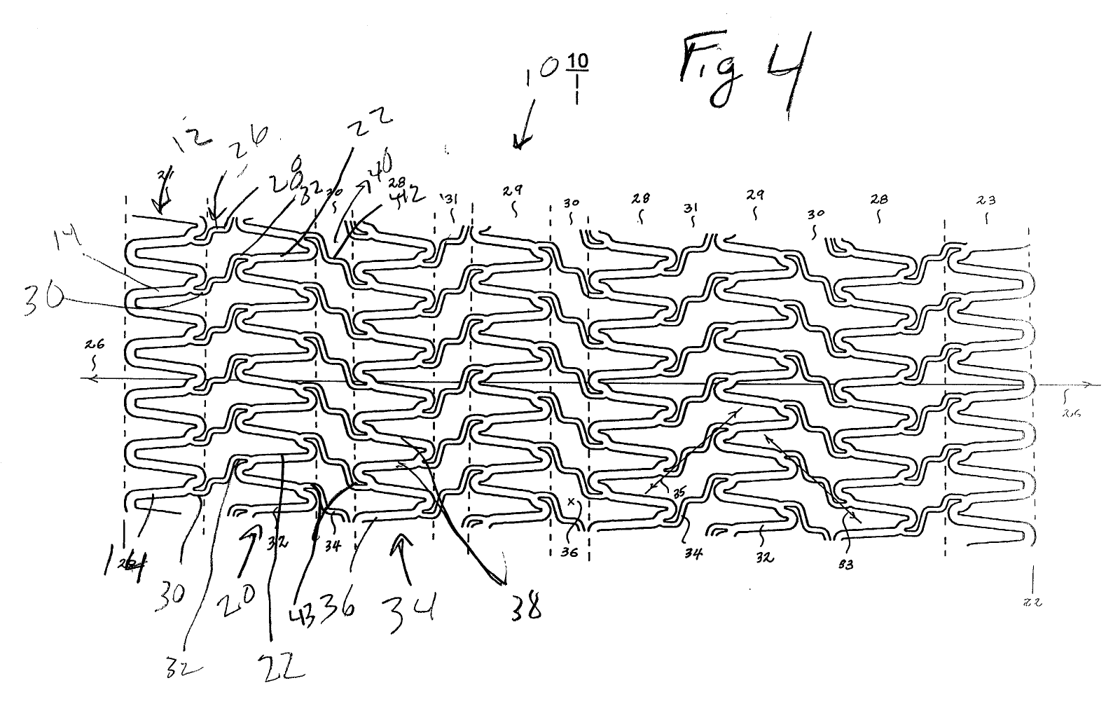

As illustrated in FIG. 4, stent 10 includes a first connecting strut column 26 with a plurality of individual connecting struts 28. Each connecting strut 28 is an extension arm 30 of an expansion strut 14 from first expansion column 12, and an extension arm 32 of an expansion strut 22 of second expansion column 20.

In the FIG. 4 embodiment, expansion columns 12, 20, 34, and the others in stent 10, have six zigzag cycles. Each six cycle expansion column includes twelve horizontally arranged expansion struts Connecting columns 26, 40 and the others do not have a zigzag cycle, but have doulbe stair-step connecting struts that are arranged in a paralleling alignment. For every one pair of expansion struts, there is only one associated connecting strut and the ratio of expansion struts to connecting struts is two-one.

Stent 10 also includes additional expansion and connecting strut columns. Stent 10 includes a third expansion column 34 with expansion struts 36 that form expansion strut pairs 38. A second connecting strut column 40 is made of a plurality of individual connecting struts 42. Each connecting strut 42 is an extension arm 43 of an expansion strut 22 from second expansion column 20 and an extension arm 44 of an expansion strut 36 of third expansion column 34.

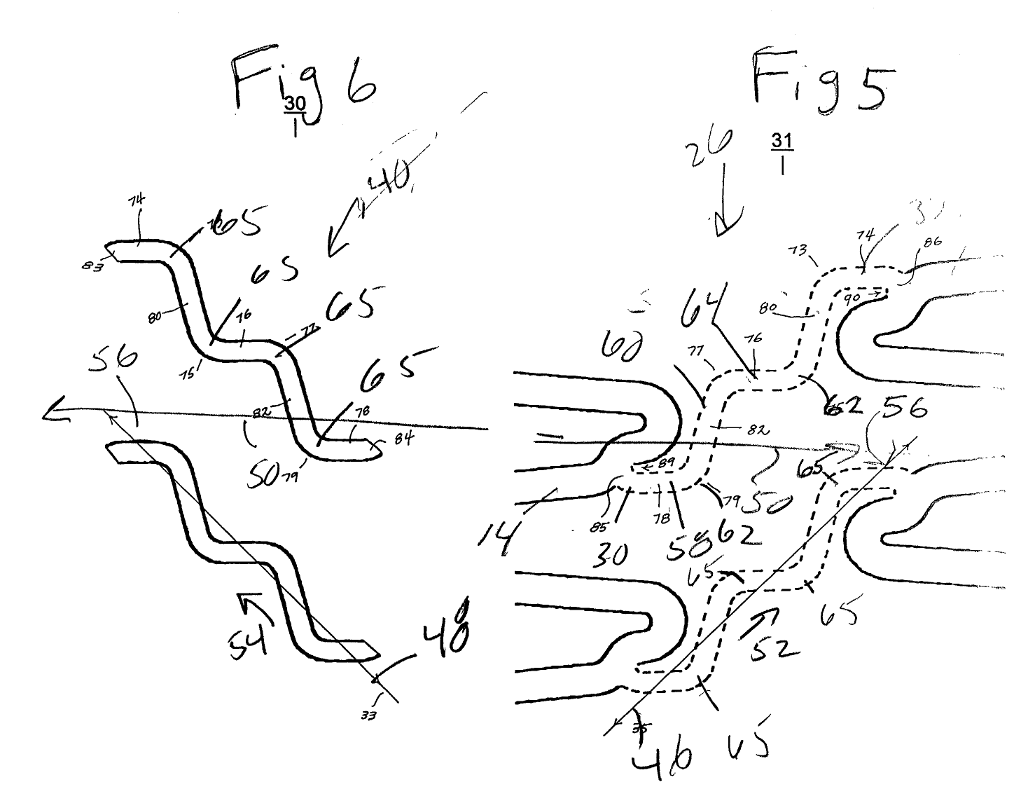

In various embodiments, one or both extension arms 30 and 32 extend from contra-lateral sides or ipsi-lateral sides of two opposing expansion strut pairs 16 and 24 (FIG. 5); and one or both extension arms 32 and 46 extend from contra-lateral sides or ipsi-lateral sides of two opposing expansion strut pairs 24 and 38 respectively. Extension from contra-lateral sides provides a diagonal link pathway and multiple angled pivot points of a connecting strut 28 and 42 provides enhanced flexibility, conformability and excellent crimping characteristics to stent 10.

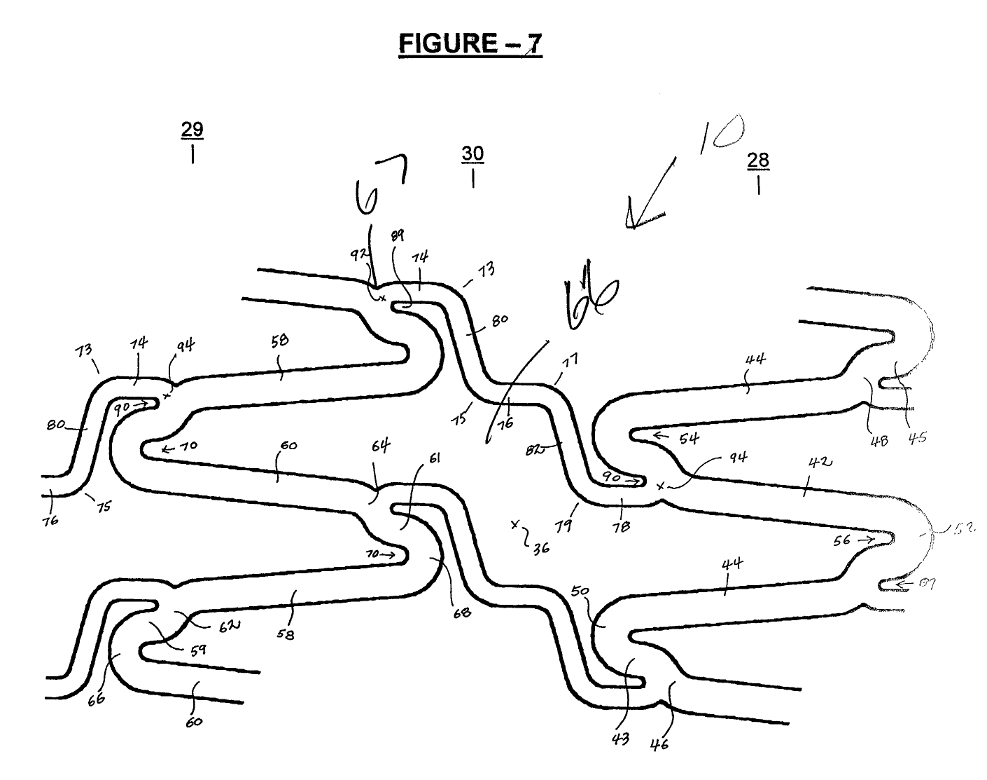

Connecting struts 28 in first connecting strut column 26 have a longitudinal axis 46 (FIG. 5) and connecting struts 42 in second connecting strut column 40 have a longitudinal axis 48 (FIG. 6) that is non-parallel to longitudinal axis 46. In various embodiments, longitudinal axis 46 and 48 can be, (i) non-perpendicular to a longitudinal axis 50 of stent 10, (ii) substantially perpendicular to longitudinal axis 50, (iii) substantially diagonal in angle with respect to longitudinal axis 50 and (iv) substantially parallel to longitudinal axis 50.

Longitudinal axis 46 extends in one direction 52 while longitudinal axis 48 extends in an opposite direction 54. Longitudinal axis 46 and 48 each have a diagonal angle 56 with respect to a longitudinal axis of stent 10. Diagonal angle 56 of longitudinal axis 46 extends in direction 52 in any number of different patterns, while diagonal angle 56 of longitudinal axis 48 extends in direction 54 in any number of different patterns. Substantially all of the connecting struts 28 in first connecting strut column 26 have a parallel longitudinal axi 46. The same is true with every connecting strut 42 in second connecting strut column 40, as well as other connecting struts in other connecting strut columns. Preferablly, every connecting strut 42 in first connecting strut column 26 has the same diagonal angle 56 with respect to longitudinal axis 50. The same is true of all other connecting struts in the other connecting strut columns of stent 10. Each longitudinal axis 46 and 48 has a slant angle vertical configuration, e.g., with diagonal angle 56, relative to longiduinal axis 50 of stent 10. This slant angle vertical configuration enhances the flexibility of stent 10 and is crimping characteristics on a balloon.

In various embodiments of the present invention, connecting struts 28, 42, and any other connecting struts in additional connecting strut columns form a, (i) single stair-step pattern, (ii) double stair-step pattern (FIGS. 5 and 6), (iii) multiple stair-step pattern, (iv) a stair-step pattern that includes at least one substantially horizontal segment 58 and at least one substantially slant-angled segment 60 and (v) at least one substantially horizontal segment 58, at least one substantially slant-angled segment 60 and a curved section 62 that joins substantially horizontal segment 58 with substantially slant angled segment 60 (vi) first segment 58 and a second segment 64, with at least a portion of first segment 58 is positioned in close proximity to a loop of an expansion strut pair 16 in first expansion column 12. Curved section 62 can have one radius of curvature, multiple radii of curvature, variabel degrees radius or radii or curvature, a wide or a narrow radius of curvature.

In the FIGS. 5 and 6 embodiments, expansion struts 14 and 22 have double stair-step patterns with multiple angled pivot points 65. Pivot points 65 also enhance the flexibility of stent 10. Additionally, linking first and second expansion columns 12 and 20 in a diagonally manner relative to longitudianl axis 50 provides further flexibility to stent 10.

Adjacent expansion columns and their associated connecting strut columns define a plurality of cells 66 that are illustrated in FIG. 7. Cells 66 have asymmetrical or symmetrical geometries. Cells 66 can have evenly spaced geometric shapes throughout stent 10. In one embodiment, cells 66 have substantially six sides when stent 10 is in a nominally expanded state. In another embodiment, cells 66 have substantially hexagonal geometric configurations when stent 10 is in a nominally expanded state. Optionally included are strain relief notches 67 that relieve the strain caused by metal deformation when stent 10 is expanded in the deployment phase.

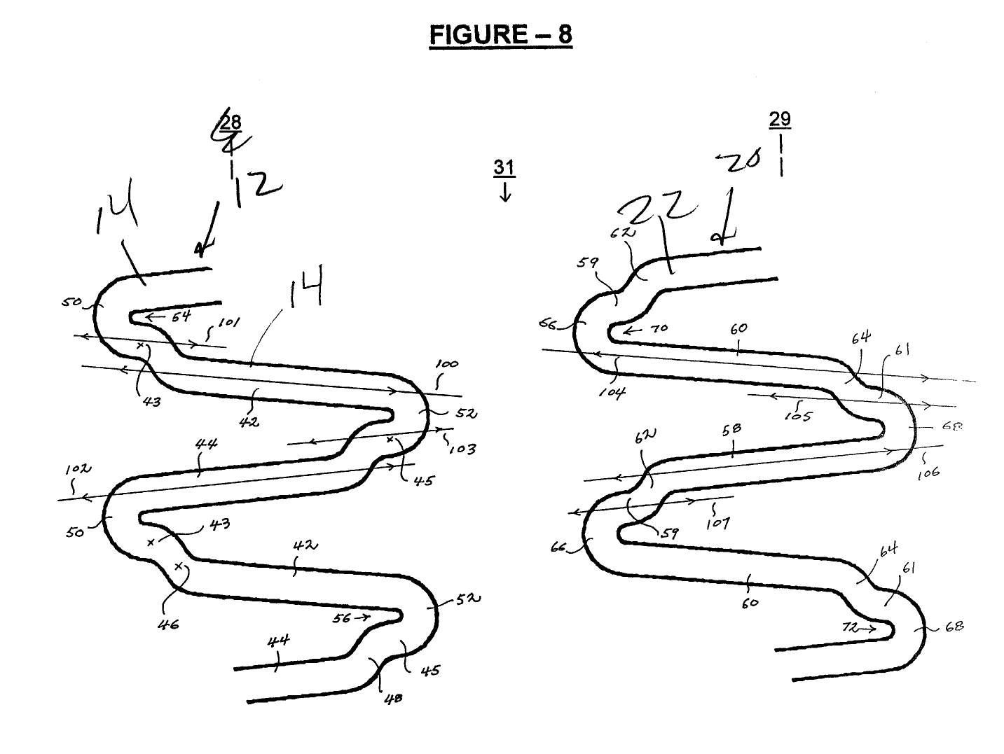

FIG. 8 illustrates one embodiment of the spacing and alignment characteristics of expansion columns 12 and 20 without illustrating first connecting strut column 26. In this embodiment, the width of first connecting strut column 26 is narrower than the width of expansion columns 12 and 20. However, the width of first connecting strut column 26 can be narrower, the same as or wider than the width of expansion columns 12 and 20. Additionally, the width of any connecting strut column in stent 10 can be variable and different from one or more of the other connecting strut columns of stent 10. Further, the width of any expansion column in stent 10 can be different and variable from one or more other expansion columns in stent 10.

First, second and third expansion columns 12, 20 and 34 can each form a corrugated expansion ring.

In another embodiment, expansion strut pair 16 loops and expansion strut pair 24 loops form first and second bifurcated expansion strut arms 68 at expansion strut bifurcation points.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A stent in a non-expanded state, comprising:
a first expansion column including individual expansion struts forming a plurality of expansion strut pairs, wherein two adjacent expansion strut pairs share a common strut;
a second expansion column including individual expansion struts forming a plurality of expansion strut pairs, wherein two adjacent expansion strut pairs share a common strut;
a first connecting strut column including a plurality of individual connecting struts, wherein each of an individual connecting strut is an extension arm of an individual expansion strut from the first expansion column and an extension arm of an individual expansion strut of the second expansion column.

2. The stent of claim 1, wherein at least one end of a connecting strut is an extension arm of an expansion strut from the first or second expansion column.

3. The stent of claim 1, wherein each end of a connecting strut is an extension arm of an expansion strut, extending from contra-lateral sides of two opposing expansion strut pairs of the first and second expansion columns.

4. The stent of claim 1, wherein at least one end of a connecting strut is an extension arm of an expansion strut, extending from contra-lateral side of one of two opposing expansion strut pairs of the first and second expansion columns.

5. The stent of claim 4, wherein at least one end of a connecting strut is an extension arm of an expansion strut, extending from ipsi-lateral side of one of two opposing expansion strut pairs of the first and second expansion columns.

6. The stent of claim 1, wherein each connecting strut has a longitudinal axis that is non-perpendicular to a longitudinal axis of the stent.

7. The stent of claim 1, wherein each connecting strut has a longitudinal axis that is substantially perpendicular to a longitudinal axis of the stent.

8. The stent of claim 1, wherein each connecting strut has a longitudinal axis that is substantially diagonal in angle with respect to a longitudinal axis of the stent.

9. The stent of claim 1, wherein each connecting strut has a longitudinal axis that is substantially parallel to a longitudinal axis of the stent.

10. The stent of claim 1, wherein each connecting strut has a longitudinal axis, and substantially every longitudinal axis of a connecting strut in the first connecting column is parallel to the others in the first connecting column.

11. The stent of claim 1, further comprising:
a plurality of expansion columns coupled by a plurality of connecting strut columns, wherein each of a connecting strut in a connecting strut column has a longitudinal axis, and substantially every longitudinal axis of a connecting strut in a connecting column is parallel to the longitudinal axis of the connecting struts in that column.

12. The stent of claim 1, further comprising:
a plurality of expansion columns coupled by a plurality of connecting strut columns, wherein each of a connecting strut in a connecting strut column has a longitudinal axis, and substantially every longitudinal axis of a connecting strut in a connecting column is at a diagonal angle with respect to a longitudinal axis of the stent.

13. The stent of claim 12, wherein each longitudinal axis of a connecting strut in a connecting strut column is parallel to the longitudinal axes of other connecting strut in the same connecting strut column at a diagonal angle with respect to a longitudinal axis of the stent.

14. The stent of claim 1, wherein each connecting strut forms a double stair-step pattern.

15. The stent of claim 1, wherein each connecting strut forms more than one stair-step pattern.

16. The stent of claim 1, wherein each connecting strut forms at least one stair-step pattern.

17. The stent of claim 1, wherein each connecting strut forms at least one stair-step pattern that includes at least one substantially horizontal segment and at least one substantially slant-angled segment.

18. The stent of claim 1, wherein each connecting strut forms a stair-step pattern with at least one substantially horizontal segment, at least one substantially slant-angled segmen and a curved section that joins the substantially horizontal segment and the substantially slant angled segment, the curved section having at least one radius of curvature.

19. The stent of claim 18, wherein a radius of curvature at a junction between a substantially horizontal segment and a substantially slant-angled segment has a wide angle radius of curvature or a narrow angle radius of curvature.

20. The stent of claim 1, wherein each connecting strut forms a stair-step pattern with at least one substantially horizontal segment, at least one substantially slant-angled segmen and a curved section that joins the substantially horizontal segment and the substantially slant angled segment, the curved section having at two radii of curvature.

21. The stent of claim 1, further comprising:
a third expansion column including individual expansion struts forming a plurality of expansion strut pairs, wherein two adjacent expansion strut pairs share a common strut;
a second connecting strut column including a plurality of individual connecting struts, wherein each of an individual connecting strut is an extension arm of an individual expansion strut from the first expansion column and an extension arm of an individual expansion strut of the second expansion column.

22. The stent of claim 21, wherein each connecting strut in the first connecting strut column has a first longitudinal axis and each connecting strut in the second connecting strut column has a second longitudinal axis that is non-parallel to the first longitudinal axis.

23. The stent of claim 21, further comprising:
a plurality of expansion columns coupled by a plurality of connecting strut columns, each of a connecting strut in a connecting strut column having a longitudinal axis, wherein the longitudinal axis of the connecting struts in a connecting strut column extend in an opposite direction relative to the longitudinal axis of connecting struts in an adjacent connecting strut column.

24. The stent of claim 23, wherein the longitudinal axis of each of a connecting strut in a connecting strut column has a diagonal angle with respect to the longitudinal axis of the stent, each longitudinal axis of the connecting struts in a connecting strut column having a diagonal angle in an opposite direction relative to a diagonal angle of each longitudinal axis of the connecting strut in an adjacent connecting strut columns.

25. The stent of claim 23, wherein a diagonal angle of the longitudinal axis of each connecting strut in one connecting strut column having opposite direction in an alternating pattern in relation to a diagonal angle of the longitudinal axis of each connecting strut in an adjacent connecting strut column of a plurality of consecutive connecting strut columns.

26. The stent of claim 1, wherein the first expansion column, the second expansion column and the first connecting strut column define a plurality of cells.

27. The stent of claim 26, wherein the cells have asymmetrical geometry.

28. The stent of claim 26, wherein the cells have evenly spaced geometric shapes throughout the stent.

29. The stent of claim 26, wherein, the cells have substantially six sides when the stent is in a nominally expanded state

30. The stent of claim 26, the cells have substantially hexagonal geometric configurations when the stent is in a nominally expanded state.

31. The stent of claim 1, wherein each end of a connecting strut is an extension of an expansion strut that extends from contra-lateral sides of two opposing expansion strut pairs of the first and second expansion columns.

32. The stent of claim 1, wherein each expansion strut has at least one stair-step segment at one end of the expansion strut.

33. The stent of claim 1, wherein one expansion strut of an expansion strut pair has a stair-step segment at a proximal end and the other expansion strut of the expansion strut pair has a stair-step segment at a distal end.

34. The stent of claim 1, wherein the first and second expansion columns each forms a corrugated expansion ring.

35. The stent of claim 21, wherein the third expansion column forms a corrugated expansion ring.

36. A stent in a non-expanded state, comprising:
a first expansion column including individual expansion struts forming a plurality of expansion strut pair loops that couple adjacent individual expansion struts, wherein two adjacent expansion strut pairs share a common strut;
a second expansion column including individual expansion struts forming a plurality of expansion strut pair loops that couple adjacent individual expansion struts, wherein two adjacent expansion strut pairs share a common strut;
a first connecting strut column including a plurality of individual connecting struts, wherein each of an end of an individual connecting strut extends from contra-lateral sides of expansion strut pair loops of the first and second expansion columns.

37. The stent of claim 36, wherein the individual expansion struts of the first and second expansion column form a plurality of expansion strut pair loops that couple adjacent individual expansion struts.

38. The stent of claim 36, wherein expansion strut pair loops of the first and second expansion columns are aligned in a peak to valley geometry.

39. The stent of claim 36, wherein expansion strut pair loops of the first and second expansion columns are aligned in a peak to peak geometry.

40. The stent of claim 36, wherein the plurality of individual connecting struts of the first connecting strut column each have a first segment and a second segment, and at least a portion of the first segment is positioned in close proximity in front of an expansion strut pair loop of the first expansion column.

41. The stent of claim 36, wherein at least a portion of the second section is positioned in close proximity in front of an expansion strut pair loop of the second expansion column.

42. The stent of claim 40, wherein close proximity is a distance of at least 0.001 inch.

43. The stent of claim 40, wherein close proximity is a distance less than 0.04 inch.

44. The stent of claim 36, wherein at least one end of a connecting strut is an extension arm of an expansion strut from the first or second expansion column.

45. The stent of claim 36, wherein each end of a connecting strut is an extension arm of an expansion strut, extending from contra-lateral sides of two opposing expansion strut pairs of the first and second expansion columns.

46. The stent of claim 36, wherein at least one end of a connecting strut is an extension arm of an expansion strut, extending from contra-lateral side of one of two opposing expansion strut pairs of the first and second expansion columns.

47. The stent of claim 46, wherein at least one end of a connecting strut is an extension arm of an expansion strut, extending from ipsi-lateral side of one of two opposing expansion strut pairs of the first and second expansion columns.

48. The stent of claim 36, wherein each connecting strut has a longitudinal axis that is non-perpendicular to a longitudinal axis of the stent.

49. The stent of claim 36, wherein each connecting strut has a longitudinal axis that is substantially perpendicular to a longitudinal axis of the stent.

50. The stent of claim 36, wherein each connecting strut has a longitudinal axis that is substantially diagonal in angle with respect to a longitudinal axis of the stent.

51. The stent of claim 36, wherein each connecting strut has a longitudinal axis that is substantially parallel to a longitudinal axis of the stent.

52. The stent of claim 36, wherein each connecting strut has a longitudinal axis, and substantially every longitudinal axis of a connecting strut in the first connecting column is parallel to the others in the first connecting column.

53. The stent of claim 36, further comprising:
a plurality of expansion columns coupled by a plurality of connecting strut columns, wherein each of a connecting strut in a connecting strut column has a longitudinal axis, and substantially every longitudinal axis of a connecting strut in a connecting column is parallel to the longitudinal axis of the connecting struts in that column.

54. The stent of claim 36, further comprising:
a plurality of expansion columns coupled by a plurality of connecting strut columns, wherein each of a connecting strut in a connecting strut column has a longitudinal axis, and substantially every longitudinal axis of a connecting strut in a connecting column is at a diagonal angle with respect to a longitudinal axis of the stent.

55. The stent of claim 54, wherein each longitudinal axis of a connecting strut in a connecting strut column is parallel to the longitudinal axis of other connecting strut in the same connecting strut column at a diagonal angle with respect to a longitudinal axis of the stent.

56. The stent of claim 36, wherein each connecting strut forms a double stair-step pattern.

57. The stent of claim 36, wherein each connecting strut forms more than one stair-step pattern.

58. The stent of claim 36, wherein each connecting strut forms at least one stair-step pattern.

59. The stent of claim 36, wherein each connecting strut forms at least one stair-step pattern, wherein each stair-step pattern has at least one substantially horizontal segment and at least one substantially slant-angled segment in an unbroken fashion.

60. The stent of claim 59, wherein each connecting strut forming a stair-step pattern with at least one substantially horizontal segment and at least one substantially slant-angled segment, wherein a stair-step pattern of a connecting strut may have a radius of curvature at a junction between a substantially horizontal segment and a substantially slant-angled segment.

61. The stent of claim 60, wherein a radius of curvature at a junction between a substantially horizontal segment and a substantially slant-angled segment may have a wide angle radius of curvature or a narrow angle radius of curvature.

62. The stent of claim 56, wherein each connecting strut forms a curvilinear corrugated pattern as a results of adding variable degrees of radius of curvature to a stair-step pattern.

63. The stent of claim 56, wherein each connecting strut forms a curvilinear corrugated pattern as a results of adding variable degrees of radius of curvature to the junction between the substantially horizontal segment and the substantially slant-angled segment.

64. The stent of claim 36, further comprising:
a third expansion column including individual expansion struts forming a plurality of expansion strut pair loops that couple adjacent individual expansion struts, wherein two adjacent expansion strut pairs share a common strut;
a second connecting strut column including a plurality of individual connecting struts, wherein each of an end of an individual connecting strut extends from contra-lateral sides of expansion strut pair loops of the first and second expansion columns.

65. The stent of claim 64, further comprising:
a plurality of expansion columns coupled by a plurality of connecting strut columns, each of a connecting strut in a connecting strut column having a longitudinal axis, wherein the longitudinal axis of the connecting struts in a connecting strut column extend in an opposite direction relative to the longitudinal axis of connecting struts in an adjacent connecting strut column.

66. The stent of claim 65, wherein the longitudinal axis of each of a connecting strut in a connecting strut column has a diagonal angle with respect to the longitudinal axis of the stent, each longitudinal axis of the connecting struts in a connecting strut column having a diagonal angle in an opposite direction relative to a diagonal angle of each longitudinal axis of the connecting strut in an adjacent connecting strut columns.

67. The stent of claim 65, wherein a diagonal angle of the longitudinal axis of each connecting strut in one connecting strut column having opposite direction in an alternating pattern in relation to a diagonal angle of the longitudinal axis of each connecting strut in an adjacent connecting strut column in a plurality of consecutive connecting strut columns.

68. The stent of claim 36, wherein the first expansion column, the second expansion column and the first connecting strut column define a plurality of cells.

69. The stent of claim 68, wherein the cells have asymmetrical geometries.

70. The stent of claim 68, wherein the cells have evenly spaced geometric shapes throughout the stent.

71. The stent of claim 68, wherein, the cells have substantially six sides when the stent is in a nominally expanded state.

72. The stent of claim 68, the cells have substantially hexagonal geometric configurations when the stent is in a nominally expanded state.

73. The stent of claim 36, wherein each end of a connecting strut is an extension of an expansion strut that extends from contra-lateral sides of two opposing expansion strut pairs of the first and second expansion columns.

74. The stent of claim 36, wherein each expansion strut has at least one stair-step segment at one end of the expansion strut.

75. The stent of claim 36, wherein one expansion strut of an expansion strut pair has a stair-step segment at a proximal end and the other expansion strut of the expansion strut pair has a stair-step segment at a distal end.

76. The stent of claim 36, wherein the first and second expansion columns each form a corrugated expansion ring.

77. The stent of claim 36, wherein each end of a connecting strut is an extension of an expansion strut that extends from contra-lateral sides of two opposing expansion strut pairs of the first and second expansion columns.

78. A stent in a non-expanded state, comprising:
a first expansion column including individual expansion struts forming a plurality of expansion strut pair loops that couple adjacent individual expansion struts, wherein two adjacent expansion strut pairs share a common strut;
a second expansion column including individual expansion struts forming a plurality of expansion strut pair loops that couple adjacent individual expansion struts, wherein two adjacent expansion strut pairs share a common strut;
a first connecting strut column including a plurality of individual connecting struts, wherein each of an end of an individual connecting strut extends from ipsi-lateral sides of expansion strut pair loops of the first and second expansion columns.

79. A stent in a non-expanded state, comprising:
a first expansion column including individual expansion struts each having a stair-step segment at one end, the individual expansion struts forming a plurality of expansion strut pairs with a loop that couples adjacent individual expansion struts to form an expansion strut pair, wherein two adjacent expansion strut pairs share a common strut;
a second expansion column including individual expansion struts each having a stair-step segment at one end, the individual expansion struts forming a plurality of expansion strut pairs with a loop that couples adjacent individual expansion struts to form an expansion strut pair, wherein two adjacent expansion strut pairs share a common strut;
a first connecting strut column including a plurality of individual connecting struts each having a first segment and a second segment, wherein at least a portion of the first segment is positioned in close proximity to a loop of an expansion strut pair in the first expansion column.

80. The stent of claim 79, wherein each end of a connecting strut is an extension of an expansion strut that extends from contra-lateral sides of two opposing expansion strut pairs of the first and second expansion columns.

81. The stent of claim 79, wherein each expansion strut has at least one stair-step segment at one end of the expansion strut.

82. The stent of claim 79, wherein one expansion strut of an expansion strut pair has a stair-step segment at a proximal end and the other expansion strut of the expansion strut pair has a stair-step segment at a distal end.

83. The stent of claim 79, wherein the plurality of individual connecting struts of the first connecting strut column each have a first segment and a second segment, and at least a portion of the first segment is positioned in close proximity in front of an expansion strut pair loop of the first expansion column.

84. The stent of claim 79, wherein at least a portion of the second section is positioned in close proximity to in front of an expansion strut pair loop of the second expansion column.

85. The stent of claim 79, wherein close proximity is a distance of at least 0.001 inch.

86. The stent of claim 79, wherein close proximity is a distance less than 0.04 inch.

87. The stent of claim 79, wherein at least one end of a connecting strut is an extension arm of an expansion strut from the first or second expansion column.

88. The stent of claim 79, wherein each end of a connecting strut is an extension arm of an expansion strut, extending from contra-lateral sides of two opposing expansion strut pairs of the first and second expansion columns.

89. The stent of claim 79, wherein at least one end of a connecting strut is an extension arm of an expansion strut, extending from contra-lateral side of one of two opposing expansion strut pairs of the first and second expansion columns.

90. The stent of claim 89, wherein at least one end of a connecting strut is an extension arm of an expansion strut, extending ipsi-lateral side of one of two opposing expansion strut pairs of the first and second expansion columns.

91. The stent of claim 79, wherein each connecting strut has a longitudinal axis that is non-perpendicular to a longitudinal axis of the stent.

92. The stent of claim 79, wherein each connecting strut has a longitudinal axis that is substantially perpendicular to a longitudinal axis of the stent.

93. The stent of claim 79, wherein each connecting strut has a longitudinal axis that is substantially diagonal in angle with respect to a longitudinal axis of the stent.

94. The stent of claim 79, wherein each connecting strut has a longitudinal axis that is substantially parallel to a longitudinal axis of the stent.

95. The stent of claim 79, wherein each connecting strut has a longitudinal axis, and substantially every longitudinal axis of a connecting strut in the first connecting column is parallel to the others in the first connecting column.

96. The stent of claim 79, further comprising:
a plurality of expansion columns coupled by a plurality of connecting strut columns, wherein each of a connecting strut in a connecting strut column has a longitudinal axis, and substantially every longitudinal axis of a connecting strut in a connecting column is parallel to the longitudinal axis of the connecting struts in that column.

97. The stent of claim 79, further comprising:
a plurality of expansion columns coupled by a plurality of connecting strut columns, wherein each of a connecting strut in a connecting strut column has a longitudinal axis, and substantially every longitudinal axis of a connecting strut in a connecting column is at a diagonal angle with respect to a longitudinal axis of the stent.

98. The stent of claim 97, wherein each longitudinal axis of a connecting strut in a connecting strut column is parallel to the longitudinal axes of other connecting strut in the same connecting strut column at a diagonal angle with respect to a longitudinal axis of the stent.

99. The stent of claim 79, wherein each connecting strut forms a double stair-step pattern.

100. The stent of claim 79, wherein each connecting strut forms more than one stair-step pattern.

101. The stent of claim 79, wherein each connecting strut forms at least one stair-step pattern.

102. The stent of claim 79, wherein each connecting strut forms at least one stair-step pattern, wherein each stair-step pattern has at least one substantially horizontal segment and at least one substantially slant-angled segment in an unbroken fashion.

103. The stent of claim 102, wherein each connecting strut forming a stair-step pattern with at least one substantially horizontal segment and at least one substantially slant-angled segment, wherein a stair-step pattern of a connecting strut may have a radius of curvature at a junction between a substantially horizontal segment and a substantially slant-angled segment.

104. The stent of claim 103, wherein a radius of curvature at a junction between a substantially horizontal segment and a substantially slant-angled segment may have a wide angle radius of curvature or a narrow angle radius of curvature.

105. The stent of claim 98, wherein each connecting strut forms a curvilinear corrugated pattern as a results of adding variable degrees of radius of curvature to a stair-step pattern.

106. The stent of claim 98, wherein each connecting strut forms a curvilinear corrugated pattern as a results of adding variable degrees of radius of curvature to the junction between the substantially horizontal segment and the substantially slant-angled segment.

107 The stent of claim 79, further comprising:
a third expansion column including individual expansion struts each having a stair-step segment at one end, the individual expansion struts forming a plurality of expansion strut pairs with a loop that couples adjacent individual expansion struts to form an expansion strut pair, wherein two adjacent expansion strut pairs share a common strut;
a second connecting strut column including a plurality of individual connecting struts each having a first segment and a second segment, wherein at least a portion of the first segment is positioned in close proximity to a loop of an expansion strut pair in the first expansion column.

108. The stent of claim 106, wherein each connecting strut in the first connecting strut column has a first longitudinal axis and each connecting strut in the second connecting strut column has a second longitudinal axis that is non-parallel to the first longitudinal axis.

109. The stent of claim 106, further comprising:
a plurality of expansion columns coupled by a plurality of connecting strut columns, each of a connecting strut in a connecting strut column having a longitudinal axis, wherein the longitudinal axis of the connecting struts in a connecting strut column extend in an opposite direction relative to the longitudinal axis of connecting struts in an adjacent connecting strut column.

110. The stent of claim 108, wherein the longitudinal axis of a connecting strut of a connecting strut column has a diagonal angle with respect to the longitudinal axis of the stent, each longitudinal axis of the connecting struts in a connecting strut column having a diagonal angle in an opposite direction relative to a diagonal angle of the longitudinal axes of the connecting strut in an adjacent connecting strut columns.

111. The stent of claim 108, wherein a diagonal angle of the longitudinal axis of each connecting strut in one connecting strut column having opposite direction in an alternating pattern in relation to a diagonal angle of the longitudinal axis of each connecting strut in an adjacent connecting strut column of a plurality of consecutive connecting strut columns.

112. The stent of claim 79, wherein the first expansion column, the second expansion column and the first connecting strut column define a plurality of cells.

113. The stent of claim 111, wherein the cells have asymmetrical geometries.

114. The stent of claim 111, wherein the cells have evenly spaced geometric shapes throughout the stent.

115. The stent of claim 111, wherein, the cells have substantially six sides when the stent is in a nominally expanded state.

116. The stent of claim 111, the cells have substantially hexagonal geometric configurations when the stent is in a nominally expanded state.

117. The stent of claim 79, wherein the first and second expansion columns each form a corrugated expansion ring.

118. A stent in a non-expanded state, comprising:
a first expansion column including individual expansion struts forming a plurality of expansion strut pair loops that form first and second bifurcated expansion strut arms at expansion strut bifurcation points, wherein two adjacent expansion strut pairs share a common strut;
a second expansion column including individual expansion struts forming a plurality of expansion strut pair loops that form first and second bifurcated expansion strut arms at expansion strut bifurcation points, wherein two adjacent expansion strut pairs share a common strut;
a first connecting strut column including a plurality of individual connecting struts, wherein, one of the expansion strut arms forms a portion of a connecting strut.

119. The stent of claim 118, wherein at least one end of a connecting strut is an extension arm of an expansion strut from the first or second expansion column.

120. The stent of claim 118, wherein each end of a connecting strut is an extension arm of an expansion strut, extending from contra-lateral sides of two opposing expansion strut pairs of the first and second expansion columns.

121. The stent of claim 118, wherein at least one end of a connecting strut is an extension arm of an expansion strut, extending from contra-lateral side of one of two opposing expansion strut pairs of the first and second expansion columns.

122. The stent of claim 121, wherein at least one end of a connecting strut is an extension arm of an expansion strut, extending from ipsi-lateral side of one of two opposing expansion strut pairs of the first and second expansion columns.

123. The stent of claim 118, wherein each connecting strut has a longitudinal axis that is non-perpendicular to a longitudinal axis of the stent.

124. The stent of claim 118, wherein each connecting strut has a longitudinal axis that is substantially perpendicular to a longitudinal axis of the stent.

125. The stent of claim 118, wherein each connecting strut has a longitudinal axis that is substantially diagonal in angle with respect to a longitudinal axis of the stent.

126. The stent of claim 118, wherein each connecting strut has a longitudinal axis that is substantially parallel to a longitudinal axis of the stent.

127. The stent of claim 118, wherein each connecting strut has a longitudinal axis, and substantially every longitudinal axis of a connecting strut in the first connecting column is parallel to the others in the first connecting column.

128. The stent of claim 118, further comprising:
a plurality of expansion columns coupled by a plurality of connecting strut columns, wherein each of a connecting strut in a connecting strut column has a longitudinal axis, and substantially every longitudinal axis of a connecting strut in a connecting column is parallel to the longitudinal axis of the connecting struts in that column.

129. The stent of claim 118, further comprising:
a plurality of expansion columns coupled by a plurality of connecting strut columns, wherein each of a connecting strut in a connecting strut column has a longitudinal axis, and substantially every longitudinal axis of a connecting strut in a connecting column is at a diagonal angle with respect to a longitudinal axis of the stent.

130. The stent of claim 129, wherein each longitudinal axis of a connecting strut in a connecting strut column is parallel to the longitudinal axes of other connecting strut in the same connecting strut column at a diagonal angle with respect to a longitudinal axis of the stent.

131. The stent of claim 118, wherein each connecting strut forms a double stair-step pattern.

132. The stent of claim 118, wherein each connecting strut forms more than one stair-step pattern.

133. The stent of claim 118, wherein each connecting strut forms at least one stair-step pattern.

134. The stent of claim 118, wherein each connecting strut forms at least one stair-step pattern, wherein each stair-step pattern has at least one substantially horizontal segment and at least one substantially slant-angled segment in an unbroken fashion.

135. The stent of claim 134, wherein each connecting strut forming a stair-step pattern with at least one substantially horizontal segment and at least one substantially slant-angled segment, wherein a stair-step pattern of a connecting strut may have a radius of curvature at a junction between a substantially horizontal segment and a substantially slant-angled segment.

136. The stent of claim 135, wherein a radius of curvature at a junction between a substantially horizontal segment and a substantially slant-angled segment may have a wide angle radius of curvature or a narrow angle radius of curvature.

137. The stent of claim 131, wherein each connecting strut forms a curvilinear corrugated pattern as a results of adding variable degrees of radius of curvature to a stair-step pattern.

138. The stent of claim 131, wherein each connecting strut forms a curvilinear corrugated pattern as a results of adding variable degrees of radius of curvature to the junction between the substantially horizontal segment and the substantially slant-angled segment.

139. The stent of claim 118, further comprising:
a third expansion column including individual expansion struts forming a plurality of expansion strut pair loops that form first and second bifurcated expansion strut arms at expansion strut bifurcation points, wherein two adjacent expansion strut pairs share a common strut;
a second connecting strut column including a plurality of individual connecting struts, wherein, one of the expansion strut arms forms a portion of a connecting strut.

140. The stent of claim 139, wherein each connecting strut in the first connecting strut column has a first longitudinal axis and each connecting strut in the second connecting strut column has a second longitudinal axis that is non-parallel to the first longitudinal axis.

141. The stent of claim 139, further comprising:
a plurality of expansion columns coupled by a plurality of connecting strut columns, each of a connecting strut in a connecting strut column having a longitudinal axis, wherein the longitudinal axis of the connecting struts in a connecting strut column extend in an opposite direction relative to the longitudinal axis of connecting struts in an adjacent connecting strut column.

142. The stent of claim 141, wherein the longitudinal axis of each of a connecting strut of a connecting strut column has a diagonal angle with respect to the longitudinal axis of the stent, each longitudinal axis of the connecting struts in a connecting strut column having a diagonal angle in an opposite direction relative to a diagonal angle of the longitudinal axes of the connecting strut in an adjacent connecting strut columns.

143. The stent of claim 141, wherein a diagonal angle of the longitudinal axis of each connecting strut in one connecting strut column having opposite direction in an alternating pattern in relation to a diagonal angle of the longitudinal axis of each connecting strut in an adjacent connecting strut column of a plurality of consecutive connecting strut columns.

144. The stent of claim 118, wherein the first expansion column, the second expansion column and the first connecting strut column define a plurality of cells.

145. The stent of claim 144, wherein the cells have asymmetrical geometries.

146. The stent of claim 144, wherein the cells have evenly spaced geometric shapes throughout the stent.

147. The stent of claim 144, wherein, the cells have substantially six sides when the stent is in a nominally expanded state.

148. The stent of claim 144, wherein the cells have substantially hexagonal geometric configurations when the stent is in a nominally expanded state.

149. The stent of claim 118, wherein each end of a connecting strut is an extension of an expansion strut that extends from contra-lateral sides of two opposing expansion strut pairs of the first and second expansion columns.

150. The stent of claim 118, wherein each expansion strut has at least one stair-step segment at one end of the expansion strut.

151. The stent of claim 118, wherein one expansion strut of an expansion strut pair has a stair-step segment at a proximal end and the other expansion strut of the expansion strut pair has a stair-step segment at a distal end.

152. The stent of claim 118, wherein the first and second expansion columns each form a corrugated expansion ring.

153. The stent of claim 118, wherein the individual expansion struts of the first and second expansion column form a plurality of expansion strut pair loops that couple two adjacent individual expansion struts.

154. The stent of claim 153, wherein expansion strut pair loops of the first and second expansion columns are aligned in a peak to valley geometry.

155. The stent of claim 153, wherein expansion strut pair loops of the first and second expansion columns are aligned in a peak to peak geometry.
